(19) United States
(12) Patent Application Publication
(10) Pub. No.: US 2025/0209836 A1
(43) Pub. Date: Jun. 26, 2025

(54) SYSTEMS AND METHODS FOR UNIFORM OBLIQUE ILLUMINATION FOR OPTICAL IMAGING

(71) Applicant: IDEXX Laboratories, Inc., Westbrook, ME (US)

(72) Inventors: Michael MOON, Scarborough, ME (US); Jason KARP, Portland, ME (US)

(73) Assignee: IDEXX Laboratories, Inc., Westbrook, ME (US)

(21) Appl. No.: 18/989,710

(22) Filed: Dec. 20, 2024

Related U.S. Application Data
(60) Provisional application No. 63/614,150, filed on Dec. 22, 2023.

(51) Int. Cl. G06V 20/69; G01N 33/49

(52) U.S. Cl. CPC G06V 20/693; G01N 33/49; G06V 20/698

(57) ABSTRACT

A digital microscope and a method to use the same include an imaging sensor defining a microscope optical axis, a light source spaced apart from the imaging sensor, a diffuser lens optically coupled to the light source, a mask optically coupled to the diffuser, a sample holder to hold a cartridge positioned between the imaging sensor and the light source, and a controller communicatively coupled to the imaging sensor. The mask restricts the passage of light beams outside of a range between about 8 degrees and about 40 degrees with respect to the microscope optical axis. The controller includes a processor and a memory comprising instructions that, when executed by the processor, cause the processor to capture a plurality of images of a liquid sample in the cartridge with the imaging sensor, and identify one or more constituents within the liquid sample based on the plurality of images.

### CROSS REFERENCE TO RELATED APPLICATIONS

This application is a claims priority to co-pending U.S. Provisional Application No. 63/614,150 filed Dec. 22, 2023 and entitled “Systems and Methods for Uniform Oblique Illumination for Optical Imaging,” which is incorporated herein by reference in its entirety.

### TECHNICAL FIELD

The present specification relates to optical microscopy imaging, and more particularly, to optical microscopy imaging using oblique illumination.

### SUMMARY

In embodiments, systems and methods for using a digital microscope including a imaging sensor defining a microscope optical axis, a light source, a diffuser lens optically coupled to the light source, a mask optically coupled to the diffuser, wherein the mask restricts the passage of light beams outside of a range between about 8 degrees and about 40 degrees with respect to the microscope optical axis, a sample holder to hold a cartridge, and a controller communicatively coupled to the imaging sensor. The controller includes a processor and a memory including instructions that, when executed by the processor, cause the processor to capture a plurality of images of a liquid sample in the cartridge with the imaging sensor, and identify one or more cell types based on the plurality of images.

In some embodiments, the liquid sample may include blood. The liquid sample may include fine needle aspirate. The liquid sample may include ear wax sample. The liquid sample may include bacteria. In some embodiments, the processor may be configured to further identify the one or more cell types by applying a machine-learning algorithm. The processor may be configured to further identify a sample type of the liquid sample, and select the machine-learning algorithm based on the identified sample type.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 schematically depicts a side view of an oblique illumination imaging system, according to one or more embodiments shown and described herein;

FIG. 2A schematically depicts a cross-sectional side view of an asymmetric mask of the oblique illumination imaging system, according to one or more embodiments shown and described herein;

FIG. 2B schematically depicts a top view of the asymmetric mask of the oblique illumination imaging system, according to one or more embodiments shown and described herein;

FIG. 3A schematically depicts a microscopy image taken without using the asymmetric mask, according to one or more embodiments shown and described herein; and

FIG. 3B schematically depicts a microscopy image taken using the asymmetric mask, according to one or more embodiments shown and described herein.

### DETAILED DESCRIPTION

Microscopy is an important diagnostic tool for early identification and intervention of medical conditions. Cytology, for example involves the study of cell morphology, which can be utilized to identify various conditions. Additionally, the microscopic interrogation of biological samples can identify the presence of bacteria and other sources of illness.

Historically, biological samples have been examined under a microscope by preparing a dry slide of the biological sample prior to viewing and/or analyzing the dried sample. When technicians manually prepare the sample for testing, the technician typically places a liquid sample on a slide, and then manually spreads the sample across the slide. After the liquid sample has been prepared and dried, stains can be applied to the sample to assist in distinguishing different cells. Variation in preparation technique can impact the consistency, physical attributes, homogeneity, and other characteristics of the sample. Moreover, in many contexts including in veterinary contexts, staff adequately trained in slide preparation may not be available.

By contrast, liquid samples (e.g., samples maintained in a fluid state) can be prepared more simply and do not require the sample to be manually spread across a slide. Accordingly, systems capable of interrogating liquid samples are desirable and can be more broadly implemented. By interrogating liquid samples, variations attributable to preparation technique can be reduced and the training/skill required to prepare samples can be reduced.

However, the interrogation of liquid samples presents several technical challenges. Transparent or below-resolution threshold objects and features appear to be at low contrast using standard brightfield illumination and are difficult to discern. Accordingly, there is a need to enhance the contrast of transparent or below-resolution threshold objects. Embodiments according to the present disclosure use oblique angles of illumination to enhance the contrast and collect information about the three-dimensional structure of the samples in imaging cellular features.

The present disclosure pertains to systems and methods for optical imaging using uniform oblique illumination by transferring a single light source into scattered light sources with adjustable illumination angles. It is challenging to image microscopic objects and features that are either transparent or fall below the resolution threshold of microscope optics. In standard brightfield illumination, these features exhibit low contrast. Alternative illumination methods, such as dark field, phase contrast, or Dodt illumination, resolve these features at the cost of multiple additional components, leading to increased costs and complexity.

The systems and methods disclosed herein provide a cost-effective illumination scheme suitable for both brightfield imaging and the resolution of transparent, sub-resolution objects and features, particularly in the context of cell pathology. The oblique illumination system does not need complex components, such as tunable lenses or spatial light modulators.

The oblique illumination system can enhance contrast by revealing details that might be obscured under direct illumination. The varying angles of illumination provide different perspectives, allowing for improved resolution and the detection of fine structures. The adjustability of the illumination angles of the oblique illumination system allows for the acquisition of images at different depths within the sample. This provides information about the three-dimensional structure of the specimen, which is desirable for studying biological samples with complex architectures. The oblique illumination system may reduce shadowing artifacts that may occur with direct illumination.

Various embodiments of the methods and systems for clinical procedure training are described in more detail herein. Whenever possible, the same reference numerals will be used throughout the drawings to refer to the same or like parts.

As used herein, the singular forms “a,” “an” and “the” include plural referents unless the context clearly dictates otherwise. Thus, for example, reference to “a” component includes aspects having two or more such components unless the context clearly indicates otherwise.

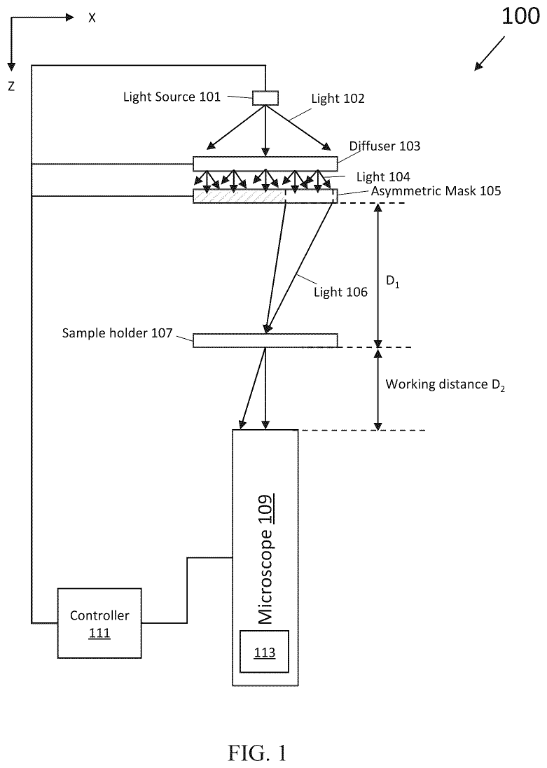

Turning to figures, FIG. 1 depicts an example oblique illumination imaging system 100. The oblique illumination imaging system 100 includes a light source 101, a diffuser 103, an asymmetric mask 105, a sample holder 107, and a microscope system 109 and a controller 111, which may be communicatively coupled to each other through one or more connections. The microscope system 109 may include a imaging sensor 113, defining a microscope optical axis. The sample holder 107 may be, without limitations, a cartridge, a microslide, a lab-on-chip, or any containers for bio-microscopy imaging. The sample holder 107 may contain a liquid sample including a solution such as blood. In embodiments, the sample holder 107 is positioned above the microscope system 109 to be imaged. The solution may include biological samples, uninterested particles, or fine needle aspirate.

The microscope system 109 may be, without limitations, an optical microscopy device, a light microscopy device, a confocal microscopy device, a multiphoton microscopy device, or a fluorescence microscopy device. In some embodiments, the microscope 109 is a digital microscope. The microscope 109 is communicatively connected to the controller 111.

The light source 101, without limitations, may include continuous spectrum light sources, multichromatic light sources, single-wavelength light sources, and fluorescence light sources of various wavelengths. The light source 101 is communicatively connected to the controller 111.

The diffuser 103 is a light scattering device, such as glass, that may scatter light 102 emitted from the light source 101 into scattered light 104. The asymmetric mask 105, which is optically coupled to the diffuser 103, may further partially block the scattered light 104 and allow controlled-asymmetric-range light 106 to be cast on the sample in the sample holder 107.

In some embodiments, the mask 105 is provided between the diffuser 103 and the microscope 109. In some embodiments, the mask 105 is between the light source 101 and the diffuser 103. In some embodiments, the diffuser 103 may directly contact the asymmetric mask 105. In some embodiments, the diffuser 103 may not be in physical contact with the asymmetric mask 105 but maintain a distance between the diffuser 103 and the asymmetric mask 105. The diffuser 103 and the asymmetric mask 105 are communicatively connected to the controller.

The diffuser 103 is optically coupled to the light source 101 and the asymmetric mask 105 is optically coupled to the diffuser 103. The asymmetric mask 105 may restrict the passage of light 104 outside of a range between about 8 degrees and about 40 degrees with respect to the microscope optical axis defined by the light source 101 and the microscope 109.

In embodiments, the distance D₁ between the asymmetric mask 105 and the sample holder 107 may be about 17.5 mm. In some embodiments, the D₁ may be greater than or equal to 8.5 mm and less than or equal to 26.5 mm. In embodiments, the working distance D₂ between the sample holder 107 to the microscope 109 is about 8.2 mm. In some embodiments, the D₂ may be greater than or equal to 4.0 mm and less than or equal to 12.5 mm.

In operation, the sample holder 107 is positioned such that the solution in the sample holder 107 is placed along the path of the light source 101. The microscope 109 may switch between, without limitations, a bright field mode, a dark field mode, and a fluorescent mode. In the bright field mode. The diffuser 103 may receive light 102 emitted from light source 101 and scatter the light 102 into light 104. The scattering process may homogenize the angular distribution of the light source 101 and improve the consistency of the phase effects and minimize the impact of changes in light illumination patterns and mechanical misalignments. The scattered light 104 as a homogenized source may be further masked through the asymmetric mask to allow only light 106 emitted in an asymmetric range. In embodiments, the asymmetric range may be between about 8 degrees and about 40 degrees. In some embodiments, the asymmetric range may be between about 0 degrees and about 90 degrees, between about 5 degrees and about 85 degrees, between about 10 degrees and about 80 degrees, between about 15 degrees and about 75 degrees, between about 20 degrees and about 70 degrees, between about 25 degrees and about 65 degrees, between about 30 degrees and about 60 degrees, between about 35 degrees and about 55 degrees, between about 40 degrees and about 50 degrees, all inclusive of the endpoints, or between any desirable degrees. In some embodiments, the asymmetric range may be greater than 0 degrees, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or any desirable degrees. In some embodiments, the asymmetric range may be less than 0 degree, 5 degrees, 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or any desirable degrees. The asymmetric range may be desirable for the visualization of red cell central pallor, white cell vacuoles, platelets, and bacteria while at the same time minimizing diffraction artifacts that interfere with normal brightfield imaging.

One or more condenser lenses (not shown in the figure) may further cooperate with the diffuser 103 and asymmetric mask 105 to focus and direct the light 102 from the light source 101 onto the sample holder 107 including one or more biological samples or artificial particles, creating a bright and uniform background. The microscope 109 may collect the transmitted light from the sample and form an image on an eyepiece or display (not shown in the figures).

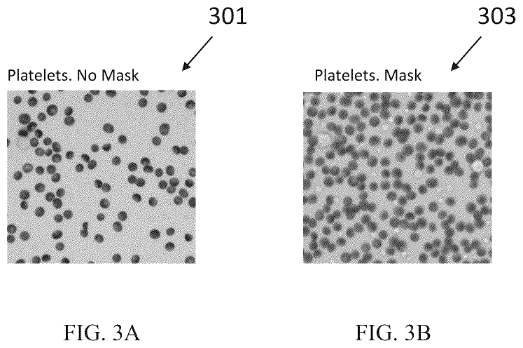

In embodiments, a biological sample in the solution in the sample holder may be analyzed by the microscope 109. An image, such as images 301 and 303 in FIGS. 3A and 3B, of the biological sample may be captured by the camera to be transmitted to the controller for automated analysis. The oblique illumination imaging system 100 may include one or more machine-learning algorithms to identify the one or more cell types. The oblique illumination imaging system 100 may further identify a sample type of the liquid sample, and select the machine-learning algorithm based on the identified sample type.

For example, as noted above, in embodiments, the system may interrogate different sample types, including and without limitation blood samples, fine needle aspirate samples, and ear swab samples. The controller 111 may include one or more machine learning programs configured to evaluate the different sample types. For example, one machine learning program configured to evaluate blood samples may evaluate images from the imaging sensor 113 and determine one or more of a red blood cell count, hematocrit, hemoglobin, mean cell volume, mean corpuscular hemoglobin, mean corpuscular hemoglobin concentration, red blood cell redistribution width, reticulocytes (percentage and number), reticulocyte hemoglobin, nucleated red blood cells, white blood cell count, neutrophils (percentage and number), lymphocytes (percentage and number), monocytes (percentage and number), eosinophils (percentage and number), basophils (percentage and number), band neutrophils, platelet count, platelet distribution width, mean platelet volume, plateletcrit, total nucleated cell count, agranulocytes (percentage and number), and granulocytes (percentage and number) within the blood sample. Another machine learning program configured to evaluate fine needle aspirate samples may evaluate images from the imaging sensor 113 and determine one or more of epithelial cells, neutrophils, lymphocytes, macrophages, plasma cells, endothelial cells, and cancererous cells in the fine needle aspirate sample. bacteria (rods or cocci), hyaline and nonhyaline casts, or crystals (bilirubin, ammonium biurate, struvite etc.). Another machine learning program, configured to evaluate ear samples may evaluate images from the imaging sensor 113 and determine one or more of bacteria (rods or cocci), yeast, ear mites, or ear ticks.

In embodiments, the system 100 selects the appropriate machine learning program based on the identified sample type (e.g., blood sample, fine needle aspirate sample, ear sample, etc.). In some embodiments, the sample type is identified via an identifier associated with the sample holder 107. The identifier may be an alpha-numeric indication, a barcode, radio-frequency identification, or the like. In some embodiments, the sample type is identified by images from the image sensor 113. For example, blood cells from a blood sample, fine needle aspirate fluid, and ear samples appear significantly different from one another. In response to identifying a predetermined quantity of blood cells in the sample, the system 100 may apply the machine learning program configured to evaluate blood samples. Similarly, in response to in response to identifying a predetermined quantity of constituents consistent with a fine needle aspirate sample, the system 100 may apply the machine learning program configured to evaluate fine needle aspirate. Likewise, in response to identifying a predetermined quantity of constituents consistent with an ear sample, the system 100 may apply the machine learning program configured to evaluate ear samples.

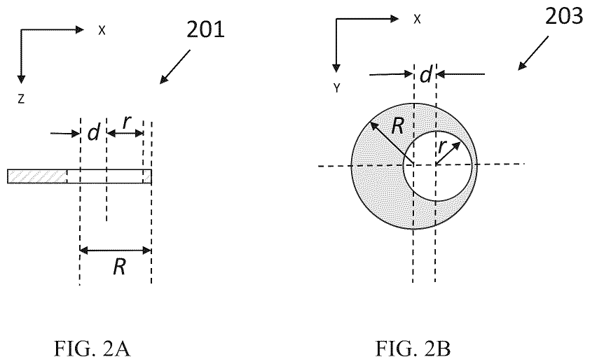

Referring to FIGS. 2A and 2B, a side view 201 (FIG. 2A) and a top view 203 (FIG. 2B) of the asymmetric mask 105 of the oblique illumination imaging system 100 are depicted, according to one embodiment. The asymmetric mask 105 may be a plate P defining an asymmetric hole h within. In the embodiment depicted in FIGS. 2A and 2B, the hole h is circumscribed by the plate P (e.g., a perimeter of the hole h is positioned entirely within a perimeter of the plate P). In some embodiments, the perimeter of the hole h intersects the perimeter of the plate P (e.g., the hole h is open-ended and the asymmetric mask 105 has a crescent-moon shape).

The radius of the asymmetric mask 105 is R. The radius of the hole is r, which is less than the R. The center of the asymmetric mask 105 may be at the optical axis relative to the light source 101, the sample holder 107, and the microscope 109. The center of the hole is not at the center of the asymmetric mask 105. The distance between the center of the asymmetric mask 105 and a center of the hole may be d, as depicted. In embodiments, the value of d may be around 3 mm. In some embodiments, the value of d may be greater than or equal to 1.5 mm and less than or equal to 4.5 mm. In embodiments, the value of r may be around 7 mm. In some embodiments, the value of r may be greater than or equal to 3.5 mm and less than or equal to 10.5 mm. In embodiments, the value of R may be greater than 5 mm, 10 mm, 15 mm, or 20 mm.

In embodiments, the asymmetric mask 105 may be a circle mask, a square mask, a rectangular mask, an oval mask, or any shape mask that may be compatible with the oblique illumination imaging system 100. The hole in the asymmetric mask 105 may be a circle hole, a square hole, a rectangular hole, an oval hole, or any shape hole that may be compatible with the oblique illumination imaging system 100.

Referring to FIGS. 3A and 3B, example microscopy images taken with (FIG. 3B) and without (FIG. 3A) using the asymmetric mask 105 are depicted. As illustrated in FIG. 3A, when the asymmetric mask 105 is not used, the acquired image provides a desirable contrast of dark cellsHowever, the red/white cells are not desirably differentiated from the background, and in the example shown in FIG. 3A, platelets are difficult to discern. Conversely, as illustrated in FIG. 3B, when the asymmetric mask 105 is used, the acquired image provides a desirable contract for both dark cells and the red/white cells, and platelets can be viewed and counted. The use of the asymmetric mask 105 of the oblique illumination imaging system 100 improves the ability to capture transparent cellular features, bacteria, and the like features.

Embodiments according to the present disclosure are described in the following numbered aspects, in which:

In a first aspect A1, the present disclosure provides a digital microscope comprising an imaging sensor defining a microscope optical axis, a light source spaced apart from the imaging sensor, a diffuser lens optically coupled to the light source, a mask optically coupled to the diffuser, wherein the mask restricts the passage of light beams outside of a range between about 8 degrees and about 40 degrees with respect to the microscope optical axis, a sample holder to hold a cartridge positioned between the imaging sensor and the light source, and a controller communicatively coupled to the imaging sensor, the controller comprising a processor and a memory comprising instructions that, when executed by the processor, cause the processor to capture a plurality of images of a liquid sample in the cartridge with the imaging sensor, and identify one or more constituents within the liquid sample based on the plurality of images.

In a second aspect A2, the present disclosure provides aspect A1, wherein the liquid sample comprises blood, and wherein identifying the one or more constituents within the liquid sample comprises identifying one or more cell types.

In a third aspect A3, the present disclosure provides aspect A2, wherein identifying one or more cell types comprises identifying a number of platelets in the sample.

In a fourth aspect A4, the present disclosure provides aspect A1, wherein the liquid sample comprises fine needle aspirate, and wherein identifying the one or more constituents within the liquid sample comprises identifying one or more cell types.

In a fifth aspect A5, the present disclosure provides aspect A1, wherein the liquid sample comprises an ear wax sample, and wherein identifying the one or more constituents within the liquid sample comprises identifying one or more of bacteria, yeast, ear mites, or ear ticks.

In a sixth aspect A6, the present disclosure provides any of the preceding aspects, wherein the instructions, when executed by the processor cause the processor to identify a sample type of the liquid sample, and select the machine-learning algorithm based on the identified sample type.

In an seventh aspect A7, the present disclosure provides any of the preceding aspects, wherein the mask is positioned between the diffuser and the sample holder.

In an eighth aspect A8, the present disclosure provides any of the preceding aspects, wherein the mask comprises a plate defining a hole.

In a ninth aspect A9, the present disclosure provides aspect A8 wherein the plate is circular.

In a tenth aspect A10, the present disclosure provides either of aspects A8 or A9, wherein the hole is circumscribed within the plate.

In an eleventh aspect A11, the present disclosure provides any of aspects A8-A10, wherein hole defines a center that is offset from a center of the plate.

In a twelfth aspect A12, the present disclosure provides aspect A11, wherein the center of the hole is positioned at least 1.5 mm from the optical axis.

In a thirteenth aspect A13, the present disclosure provides a method for evaluating a liquid sample with a digital microscope, the method comprising receiving a liquid sample on a sample holder positioned between an imaging sensor defining a microscope optical axis, and a light source, passing light from the light source through a diffuser and an asymmetric mask to the sample holder, wherein the mask restricts the passage of light beams outside of a range between about 8 degrees and about 40 degrees with respect to the microscope optical axis, capturing a plurality of images of the liquid sample with the imaging sensor, and identifying one or more constituents within the liquid sample based on the plurality of images.

In a fourteenth aspect A14, the present disclosure provides A13, wherein the liquid sample comprises blood, and wherein identifying the one or more constituents within the liquid sample comprises identifying one or more cell types.

In a fifteenth aspect A15, the present disclosure provides A14, wherein identifying one or more cell types comprises identifying a number of platelets in the sample.

In a sixteenth aspect A16, the present disclosure provides A13, wherein the liquid sample comprises fine needle aspirate, and wherein identifying the one or more constituents within the liquid sample comprises identifying one or more cell types.

In a seventeenth aspect A17, the present disclosure provides A13, wherein the liquid sample comprises an ear wax sample, and wherein identifying the one or more constituents within the liquid sample comprises identifying one or more of bacteria, yeast, ear mites, or ear ticks.

In an eighteenth aspect A18, the present disclosure provides any of A13-A17, wherein the mask is positioned between the diffuser and the sample holder.

In a nineteenth aspect A19, the present disclosure provides any of aspects A13-A18, wherein the mask comprises a plate defining a hole.

In a twentieth aspect A20, the present disclosure provides aspect A19, wherein the hole is circumscribed within the plate.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

What is claimed is:

1. A digital microscope comprising:
an imaging sensor defining a microscope optical axis;
a light source spaced apart from the imaging sensor;
a diffuser lens optically coupled to the light source;
a mask optically coupled to the diffuser, wherein the mask restricts the passage of light beams outside of a range between about 8 degrees and about 40 degrees with respect to the microscope optical axis;
a sample holder to hold a cartridge positioned between the imaging sensor and the light source; and
a controller communicatively coupled to the imaging sensor, the controller comprising a processor and a memory comprising instructions that, when executed by the processor, cause the processor to:
capture a plurality of images of a liquid sample in the cartridge with the imaging sensor; and
identify one or more constituents within the liquid sample based on the plurality of images.

2. The digital microscope of claim 1, wherein the liquid sample comprises blood, and wherein identifying the one or more constituents within the liquid sample comprises identifying one or more cell types.

3. The digital microscope of claim 2, wherein the identifying one or more cell types comprises identifying a number of platelets in the liquid sample.

4. The digital microscope of claim 1, wherein the liquid sample comprises fine needle aspirate, and wherein identifying the one or more constituents within the liquid sample comprises identifying one or more cell types.

5. The digital microscope of claim 1, wherein the liquid sample comprises an ear wax sample, and wherein identifying the one or more constituents within the liquid sample comprises identifying one or more of bacteria, yeast, ear mites, or ear ticks.

6. The digital microscope of claim 1, wherein the instructions, when executed by the processor cause the processor to identify a sample type of the liquid sample, and select a machine-learning algorithm based on the identified sample type.

7. The digital microscope of claim 1, wherein the mask is positioned between the diffuser and the sample holder.

8. The digital microscope of claim 1, wherein the mask comprises a plate defining a hole.

9. The digital microscope of claim 8, wherein the plate is circular.

10. The digital microscope of claim 8, wherein the hole is circumscribed within the plate.

11. The digital microscope of claim 8, wherein the hole defines a center that is offset from a center of the plate.

12. The digital microscope of claim 11, wherein the center of the hole is positioned at least 1.5 mm from the microscope optical axis.

13. A method for evaluating a liquid sample with a digital microscope, the method comprising:
receiving a liquid sample on a sample holder positioned between an imaging sensor defining a microscope optical axis, and a light source;
passing light from the light source through a diffuser and an asymmetric mask to the sample holder, wherein the mask restricts the passage of light beams outside of a range between about 8 degrees and about 40 degrees with respect to the microscope optical axis;
capturing a plurality of images of the liquid sample with the imaging sensor; and
identifying one or more constituents within the liquid sample based on the plurality of images.

14. The method of claim 13, wherein the liquid sample comprises blood, and wherein identifying the one or more constituents within the liquid sample comprises identifying one or more cell types.

15. The method of claim 14, wherein identifying one or more cell types comprises identifying a number of platelets in the sample.

16. The method of claim 13, wherein the liquid sample comprises fine needle aspirate, and wherein identifying the one or more constituents within the liquid sample comprises identifying one or more cell types.

17. The method of claim 13, wherein the liquid sample comprises an ear wax sample, and wherein identifying the one or more constituents within the liquid sample comprises identifying one or more of bacteria, yeast, ear mites, or ear ticks.

18. The method of claim 13, wherein the mask is positioned between the diffuser and the sample holder.

19. The method of claim 13, wherein the mask comprises a plate defining a hole.

20. The method of claim 19, wherein the hole is circumscribed within the plate.
